(19) United States
(12) Patent Application Publication
(10) Pub. No.: US 2016/0042539 A1
(43) Pub. Date: Feb. 11, 2016

(54) MULTIMEDIA READING CONTROL DEVICE, GRAPH ACQUISITION DEVICE, ELECTRONIC EQUIPMENT, GRAPH SUPPLY DEVICE AND METHODS THEREOF

(71) Applicant: SONY CORPORATION, Tokyo (JP)

(72) Inventors: Bizhong YE, Beijing (CN); Yu ZHANG, Beijing (CN); Xiaofeng FAN, Beijing (CN); Shuai LIN, Beijing (CN); Zhangbin YIN, Beijing (CN); Wenhui XIA, Beijing (CN)

(21) Appl. No.: 14/400,127

(22) PCT Filed: Aug. 29, 2014
(86) PCT No.: PCT/IB2014/064136

§ 371 Date: Nov. 10, 2014

(30) Foreign Application Priority Data
Nov. 28, 2013 (CN) 201310625545.7

(51) Int. Cl. G06T 11/20; A61B 5/08; A61B 5/11; G06T 11/60; A61B 5/0205

(52) U.S. Cl. CPC G06T 11/206; G06T 11/60; A61B 5/02055; A61B 5/11; A61B 5/0205; A61B 5/08

(57) ABSTRACT

Embodiments of the invention provide a multimedia reading control device, a graph acquisition device, an electronic equipment, a graph supply device and a method thereof, wherein the multimedia control device includes a reception unit which receives a graph of physiological parameters that varies with the progress of reading a multimedia file, a display unit which displays the graph of physiological parameters, and a response unit which reads the multimedia file in response to the progress selected according to the graph of physiological parameters. The graph of physiological parameters varying with the progress of reading the multimedia file is displayed when the multimedia file is read, so that a user selects the progress of the multimedia file according to the graph of physiological parameters, so as to read the part in the multimedia file in which the user is interested conveniently and efficiently.

### TECHNICAL FIELD

The invention relates to communication field, and particularly relates to a multimedia reading control device, a graph acquisition device, an electronic equipment, a graph supply device and a method thereof.

### CROSS REFERENCE TO RELATED APPLICATIONS

Reference is made to Chinese patent application No. 201310625545.7, filed 28 Nov. 2013, the entire disclosure of which hereby is incorporated by reference.

### BACKGROUND ART

Currently, with the development of communication technologies, electronic equipment, such as a mobile phone, a computer and the like are widely used, and one of the important functions and uses of these electronic equipment is multimedia playing. Sometimes, users need to search and select fragments in which they are interested, such as a fragment of violent plot in a movie, a high-pitched part of music and the like, when playing a multimedia file by utilizing these electronic equipment.

In a traditional method, a user drags a playing progress bar of a multimedia file and makes selections according to pictures or music of moments of playing in turn, and this type of operation is tedious and efficiency is low.

### SUMMARY

Embodiments of the invention provide a multimedia reading control device, a graph acquisition device, an electronic equipment, a graph supply device and methods thereof. A part of the multimedia file in which a user is interested can be read conveniently and efficiently.

According to a first aspect of embodiments of the invention, a multimedia reading control device is provided, which includes a first reception unit configured to receive a graph of physiological parameters that varies with the progress of reading a multimedia file, a display unit configured to display the graph of physiological parameters, and a response unit configured to read the multimedia file in response to the progress selected according to the graph of physiological parameters.

According to a second aspect of the embodiments of the invention, a graph acquisition device is provided, which includes an acquisition unit configured to acquire physiological parameters of a user when enjoying a multimedia file, so as to be used for generating a graph of physiological parameters that varies with the progress of reading the multimedia file, and a first transmission unit configured to transmit the combined graph of physiological parameters.

According to a third aspect of the embodiments of the invention, an electronic equipment is provided, which includes the multimedia reading control device according to the first aspect of the embodiments of the invention and/or the graph acquisition device according to the second aspect of the embodiments of the invention.

According to a fourth aspect of the embodiments of the invention, a graph supply device is provided, which includes a graph combination unit configured to combine at least two graphs of physiological parameters that vary with the progress of reading the multimedia file to obtain a combined graph of physiological parameters and a second transmission unit configured to transmit the combined graph of physiological parameters.

According to a fifth aspect of the embodiments of the invention, a multimedia reading control method is provided, which includes receiving a graph of physiological parameters that varies with the progress of reading a multimedia file, displaying the graph of physiological parameters, and reading the multimedia file in response to the progress selected according to the graph of physiological parameters.

According to a sixth aspect of the embodiments of the invention, a graph acquisition method is provided, which includes acquiring physiological parameters of a user when enjoying a multimedia file, so as to be used for generating a graph of physiological parameters that varies with the progress of reading the multimedia file, and transmitting the combined graph of physiological parameters.

According to a seventh aspect of the embodiments of the invention, a graph supply method is provided, which includes combining at least two graphs of physiological parameters that vary with the progress of reading the multimedia file to obtain a combined graph of physiological parameters and transmitting the combined graph of physiological parameters.

The embodiments of the invention have beneficial effects: the graph of physiological parameters varying with the progress of reading the multimedia file is displayed when the multimedia file is read, so that a user selects the progress of the multimedia file according to the graph of physiological parameters, so as to read the part in the multimedia file in which the user is interested conveniently and efficiently.

With reference to the after-mentioned description and drawings, specific embodiments of the invention are disclosed in detail, in which the ways in which the principle of the invention can be adopted are specified. It should be understood that the embodiments of the invention are not limited in the scope. The embodiments of the invention may include many variations, modifications and equivalents within the scope of the spirit of the appended claims and the provisions thereof.

Characteristics described and/or shown for one embodiment can be used in a same or similar way on one or more of other embodiments, may be combined with the characteristics in other embodiments, or replace characteristics of other embodiments.

It should be emphasized that the using of the term “comprise/include” refers to presence of characteristics, assembly, steps or components, but does not exclude presence or attachment of one or more of other characteristics, assembly, steps or components.

### BRIEF DESCRIPTION OF DRAWINGS

Many aspects of the invention can be understood better with reference to the following drawings. Parts in the drawings are not drawn proportionally, but just for showing principles of the invention. In order to facilitate showing and description of some parts of the invention, corresponding parts in the drawings may be enlarged or reduced. Elements and characteristics described in one drawing or one embodiment of the invention may be combined with elements and characteristics shown in one or more of other drawings or embodiments. In addition, in the drawings, similar reference numbers denote corresponding parts in several drawings and can be used for indicating corresponding parts used in more than one embodiment.

In the drawings:

FIG. 1 is a structural schematic of the multimedia reading control device of embodiment 1 of the invention;

FIG. 2 is a schematic of the graph of physiological parameters of embodiment 1 of the invention;

FIG. 3 is a schematic of the way in which the graph of physiological parameters of embodiment 1 of the invention is displayed;

FIG. 4 is a schematic of displaying the graph of physiological parameters when reading the movie of embodiment 1 of the invention;

FIG. 5 is a structural schematic of the response unit of embodiment 1 of the invention;

FIG. 6 is a flow chart of a method by which a response unit of embodiment 1 of the invention reads the multimedia file according to the progress selected by a user;

FIG. 7 is a structural schematic of the graph acquisition device of embodiment 2 of the invention;

FIG. 8 is a structural schematic of the electronic equipment of embodiment 3 of the invention;

FIG. 9 is a schematic block diagram of system composition of the electronic equipment of embodiment 3 of the invention;

FIG. 10 is a structural schematic of multimedia reading system of embodiment 4 of the invention;

FIG. 11 is a structural schematic of the graph supply device of embodiment 4 of the invention;

FIG. 12 is a schematic of the multimedia reading system of embodiment 4 that performs multimedia reading;

FIG. 13 is a flow chart of the multimedia reading control method of embodiment 5 of the invention;

FIG. 14 is a flow chart of the graph acquisition method of embodiment 6 of the invention;

FIG. 15 is a flow chart of the graph supply method of embodiment 7 of the invention.

### DESCRIPTION OF EMBODIMENTS

Interchangeable terms “electronic device” and “electronic equipment” include a portable radio communication device. The term “a portable radio communication device” hereinafter is referred to as “a mobile radio terminal”, “a portable electronic device” or “a portable communication device”, which includes all of the devices such as a mobile phone, a smart phone, a pager, a communication device, an electronic notebook, a person digital assistant (PDA), a smart telephone, a portable communication device and the like.

In the present application, the embodiments of the invention are described mainly concerning a portable electronic device in the form of a mobile phone (also called “cell phone”). However, it should be understood that, the invention should not be limited to the circumstance of the mobile phone, but can relate to any types of appropriate electronic equipment. Example of such electronic equipment may include a smart watch, intelligent glasses, intelligent wig, a headset device, a wearable device, a fixed-line telephone, a media player, a game device, a PDA, a computer, a digital camera and the like.

Hereinafter preferable embodiments of the invention will be described with reference to the drawings.

### Embodiment 1

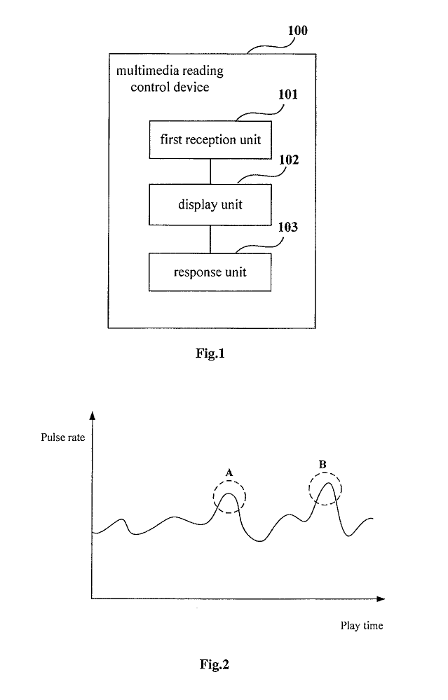

The embodiment of the invention provides a multimedia reading control device, and FIG. 1 is a structural schematic of the multimedia reading control device. As shown in FIG. 1, the multimedia reading control device 100 includes a first reception unit 101 (as used herein the term “unit” also may be understood to mean “module,” “component,” “element” and so on, as will be appreciated by those having ordinary skill in the art), a display unit 102 and a response unit 103, wherein

the first reception unit 101 is configured to receive a graph of physiological parameters that varies with the progress of reading a multimedia file;

the display unit 102 is configured to display the graph of physiological parameters;

the response unit 103 is configured to read the multimedia file in response to the progress selected according to the graph of physiological parameters.

It can be seen from the above embodiment that, the graph of physiological parameters varying with the progress of reading the multimedia file is displayed when the multimedia file is read, so that a user selects the progress of the multimedia file according to the graph of physiological parameters, so as to read the part in the multimedia file in which the user is interested conveniently and efficiently.

In this embodiment, the first reception unit 101 is used for receiving a graph of physiological parameters that varies with the progress of reading a multimedia file, wherein the graph of physiological parameters can be obtained together with the multimedia file and can be obtained separately with the multimedia file, but the embodiment of the invention is not limited thereto.

In this embodiment, the first reception unit 101 may receive the graph of physiological parameters by using any one of the existing methods, but the embodiment of the invention has no limitation to this. For example, the graph of physiological parameters can be received by using the method of downloading from a network server.

In this embodiment, the multimedia file and the physiological parameters may be any one of the existing types, but the embodiment of the invention has no limitation to the type of the multimedia file and the physiological parameters.

For example, the multimedia file may be a video file, an audio file or a text file, wherein the video file may include e.g. a movie, a TV play, a news short film, a program videotape and the like, the audio file may include e.g. a song, program recording and the like, and the text file may include e.g. an electronic book, newspaper, magazine and the like.

In this embodiment, for the video file or the audio file, the progress of reading the multimedia file may be for example the time of playing the multimedia file, and for the text file, the progress of reading the multimedia file may be for example reading chapters, paragraphs or pages of the multimedia file.

For example, the physiological parameters may be random physiological parameters that vary with emotion of a user. For example, the physiological parameters may include at least one of heartbeat, pulse, blood pressure, body temperature, a respiration parameter, and sound, a focus of eyes, pupil size, facial action, body movements and secretions.

Hereinafter exemplary description will be made by taking the multimedia file to be a video file or an audio file, and the physiological parameters to be pulse as an example.

FIG. 2 is a schematic of the graph of physiological parameters of the embodiment. As shown in FIG. 2, a horizontal ordinate denotes play time of the multimedia file, in minutes for example, and a vertical ordinate denotes pulse rate of a user, namely number of times of pulse per minute. With the multimedia file is played, emotion of a user who enjoys the multimedia file varies, so does his pulse rate. When the multimedia file is a movie, parts A and B in FIG. 2 e.g., are violent plot part of the movie, and when the multimedia file is a song, the parts A and B are e.g., a high-pitched part of the song. When the user enjoys the parts A and B, his emotion changes obviously, so does his pulse rate.

In this embodiment, when the multimedia file is a text file such as e-book, newspaper, magazine and the like, the horizontal ordinate of the graph of physiological parameters can be for example, chapter, paragraph or page of the text file such as e-book, newspaper, magazine and the like, and the vertical ordinate can be the physiological parameters. When reading different chapters, paragraphs or pages, the physiological parameters will vary, so as to form the graph of physiological parameters that varies along with the progress of reading the multimedia file. The user can select a chapter, a paragraph or a page of the text file such as e-book, newspaper, magazine and the like to read according to the graph of physiological parameters. For example, if the physiological parameters vary to a high extent when the graph of physiological parameters is displayed at chapter 3 of the e-book, the user can directly select chapter 3 to read through the contents of the e-book.

In this embodiment, the graph of physiological parameters can be obtained by using any one of the existing methods, but the embodiment of the invention is not limited to this.

For example, the graph of physiological parameters can be supplied by an issuer of the multimedia file. The issuer of the multimedia file can organize enjoyment of the multimedia file, and measures physiological parameters of viewers when they are enjoying the multimedia file at the same time, and processes the physiological parameters so as to obtain the graph of physiological parameters that varies along with the progress of reading the multimedia file.

For example, at the same time of obtaining the multimedia file, the user receives an invitation of testing the physiological parameters from the issuer of the multimedia file. After the user agrees to participate into the test, at the same time of enjoying the multimedia file, the user tests and supplies his physiological parameters to the issuer. In this way, the issuer can obtain multiple graphs of physiological parameters of multiple users, and processes the graphs in the way of e.g. combination, so as to obtain the graph of physiological parameters.

For example, culture difference may occur among users in different areas, and points of interest of users at different ages may be different as well. For this reason, graphs of physiological parameters of users in different areas or at different ages can be manufactured (e.g., made, developed or prepared) respectively according to information such as areas, ages and the like of the users, so as to supply richer and more accurate graphs of physiological parameters for the users.

For example, the graph of physiological parameters can also be supplied by a user. A movie enthusiasts spontaneously tests their physiological parameters when enjoying the multimedia file, to generate graphs of physiological parameters that each varies along with the progress of reading the multimedia file. These movie enthusiasts transmit the tested graphs of physiological parameters to a server which combines these graphs of physiological parameters and provides the combined graph to other users for use.

In this embodiment, different physiological parameters may be selected and tested for different types of multimedia files. For example, for an action movie or a science fiction movie, pulse or heartbeat of the user can be tested; for a story movie or an art movie, pupil size of the user can be tested; for a love movie, secretions such as tears of the user can be tested; and for a comedy movie, facial action or body movements of the user can be tested.

In this embodiment, two or more physiological parameters of the user can be tested at the same time to obtain graphs of different physiological parameters respectively. For example, pulse, pupil size and facial action of the user can be tested at the same time to manufacture a graph of pulse, a graph of pupil size and a graph of facial action respectively. For example, play time can be selected with reference to different graphs for different scenes in the video, wherein for a scene of violence in the video, pulse of the user changes obviously, therefore a graph of pulse is quite referential to selection of a scene of violence; for a scene of emotion communication in the video, pupil size of the user may change obviously, therefore a graph of pupil size is quite referential to selection of a scene of emotion communication; for a funny scene in the video, facial action of the user may change obviously, therefore a graph of facial action is quite referential to selection of a funny scene.

In this embodiment, physiological parameters of the user can be tested by using any one of the existing methods, but the embodiment of the invention has no limitation to this. For example, a smart watch or a smart phone can be used for testing pulse or heartbeat of the user; intelligent glasses can be used for testing pupil size, the secreted tears and action of eyelids and the like; intelligent wig can be used for testing facial action or facial expression of the user; and a device with a sound detector, e.g. an intelligent phone, a smart watch, a headset device and the like can be used for testing sound, for example, exclamations such as “wow” made by the user when watching a wonderful part.

In this embodiment, the graph of physiological parameters can be a combined graph of physiological parameters that is obtained by combining at least two graphs of physiological parameters. For example, in the above method supplied by the issuer of the multimedia file, the issuer can measure at least two viewers to obtain two or more graphs of physiological parameters and combine these graphs of physiological parameters. In addition, in the above method supplied by the user, at least two users can upload the graphs of physiological parameters to a network server which combines at least two graphs of physiological parameters.

At least two graphs of physiological parameters can be obtained by combination in any one of the existing methods, but the embodiment of the invention has no limitation to this. For example, a graph combination unit of a graph supply device in the following embodiment 4 can be used for combining at least two graphs of physiological parameters, wherein for example, the combined graph of physiological parameters can be obtained by the method of calculating a mean value, wherein for one identical progress, a mean value of physiological parameters of the progress of at least two graphs of physiological parameters is calculated, so as to generate the combined graph of physiological parameters according to the mean value of the physiological parameters of the progresses.

In this way, accuracy of the graph of physiological parameters can be improved by combining two or more graphs of physiological parameters.

In this embodiment, when the multimedia reading control device plays the multimedia file, the display unit 102 can control the display to display the graph of physiological parameters for the user to select the progress of the multimedia file according to the graph of physiological parameters. The embodiment of the invention has no limitation to display methods of the graph of physiological parameters. Hereinafter the display method of the graph of physiological parameters of the embodiment will be described exemplarily.

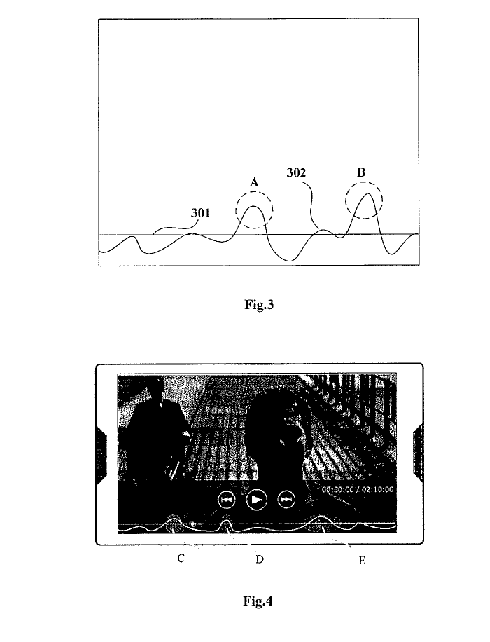

FIG. 3 is a schematic of the display method of the graph of physiological parameters of the embodiment of the invention. As shown in FIG. 3, when the multimedia file is a movie, main part of a display screen is used for displaying picture of the movie (not shown). At lower part of the display screen, a playing progress bar 301 of the movie is displayed in an existing method, and the graph of physiological parameters 302 is displayed corresponding to the playing progress bar. The parts A and B in FIG. 3 are violent plot part of the movie, such as a scene of gunfight, a scene of drag racing and the like. The user drags the playing progress bar 301 to positions corresponding to the parts A and B, or directly clicks the positions on the playing progress bar 301 that correspond to the parts A and B. In this way, it is convenient for the user to select play time of the parts A and B in which the user may be interested.

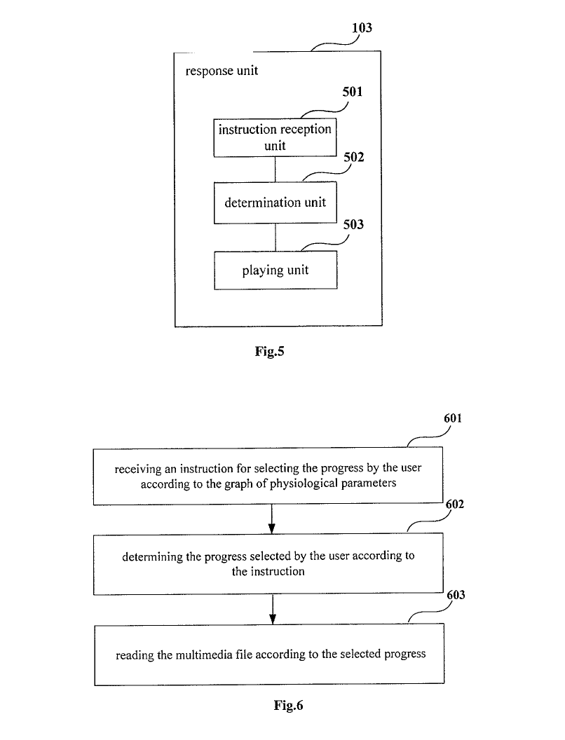

FIG. 4 is a schematic of displaying the graph of physiological parameters when the movie is played of the embodiment, and shows a scene of controlling of media playback. The graph of physiological parameters can be overlaid on the playing progress bar when the movie is played. As shown in FIG. 4, during the process of playing the movie, the graph of physiological parameters is displayed corresponding to the playing progress bar. The C, D, E areas indicate a part of drag racing, a part of gunfight and a part of a decisive battle in the movie respectively. The user can select play time of the part in which he may be interested according to the graph.

In another implementation mode of the embodiment, it is also practicable to not display the playing progress bar 301, but to use the graph of physiological parameters directly as the playing progress bar, to drag points of the graph to positions corresponding to the parts A and B by along the graph of physiological parameters or directly click the parts A and B of the graph of physiological parameters. In this way, it is convenient for the user to select play time of the parts A and B in which the user may be interested.

In another implementation mode of the embodiment, when an audio file of a song e.g. is played, the main part of the display screen can be used for displaying basic information of the song, such as lyrics, the singer, the year of publication, the issuing company and the like. Similarly, for example, the playing progress bar and the graph of physiological parameters are displayed at the lower part of the screen, or only the graph of physiological parameters is displayed in the method the same as the above, and which is not repeated herein.

In this embodiment, the graph of physiological parameters can be activated to be displayed or hidden by using any one of the existing methods, but the embodiment of the invention has no limitation to this. For example, displaying can be activated by the user's operation, such as clicking of a picture playing area on the screen during the process of playing the movie; or the displaying of the graph of physiological parameters can be hidden by another operation of the user, such as another clicking of the picture playing area on the screen.

In this embodiment, the graph of physiological parameters can be displayed during the process of normally playing the multimedia file or during the process of dragging the playing progress bar, but the embodiment of the invention has no limitation to this. In this embodiment, the response unit 103 is configured to play the multimedia file in response to the play time selected according to the graph of physiological parameters. The structure of the response unit 103 can be any one of the existing structure, but the embodiment of the invention has no limitation to the structure of the response unit.

Hereinafter the structure of the response unit will be described exemplarily.

FIG. 5 is a structural schematic of the response unit of the embodiment. As shown in FIG. 5, the response unit 103 may include an instruction receiving unit 501, a determination unit 502 and a playing unit 503, wherein

the instruction receiving unit 501 is configured to receive an instruction for selecting the progress according to the graph of physiological parameters;

the determination unit 502 is configured to determine the selected progress according to the instruction;

the playing unit 503 is configured to read the multimedia file according to the selected progress.

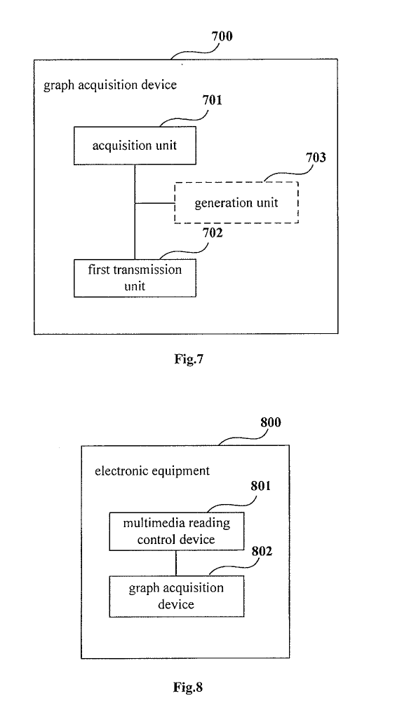

FIG. 6 is a flow chart of a method by which the response unit of the embodiment reads the multimedia file according to the progress selected by a user. As shown in FIG. 6, the method includes:

step 601: receiving an instruction of the user for selecting the progress according to the graph of physiological parameters;

step 602: determining the progress selected by the user according to the instruction;

step 603: reading the multimedia file according to the selected progress.

In this embodiment, the user can use any one of the existing methods according to the progress of selecting the graph of physiological parameters, but the embodiment of the invention has no limitation to this. For example, when the display screen is a touch screen, the user can operate on the screen directly by using a finger or a touch pen, so as to send out an instruction for selecting the progress; when the screen is a non-touch screen, the user can operate by using a mouse or a trackpad to control cursor on the screen, so as to send out an instruction for selecting the progress.

In this embodiment, it is possible to determine the progress according to the instruction and to read according to the progress in any one of the existing methods, but the embodiment of the invention has no limitation to this.

It can be seen from the above embodiment that, the graph of physiological parameters varying with the progress of reading the multimedia file is displayed when the multimedia file is read, so that a user selects the progress of the multimedia file according to the graph of physiological parameters, so as to read the part in the multimedia file in which the user is interested conveniently and efficiently.

### Embodiment 2

The embodiment of the invention provides a graph acquisition device, and FIG. 7 is a structural schematic of the graph acquisition device 700. As shown in FIG. 7, the graph acquisition device 700 includes an acquisition unit 701 and a first transmission unit 702, wherein

the acquisition unit 701 is configured to acquire physiological parameters of a user when enjoying a multimedia file, so as to be used for generating a graph of physiological parameters that varies with the progress of reading the multimedia file;

the first transmission unit 702 is configured to transmit the physiological parameters.

In this embodiment, the acquisition unit 701 may obtain the physiological parameters of the user when he enjoys the multimedia file by using any one of the existing methods, but the embodiment of the invention has no limitation to this. For example, the physiological parameters can be obtained by the method of measurement.

In this embodiment, the first transmission unit 702 may transmit the graph of physiological parameters by using any one of the existing methods, but the embodiment of the invention has no limitation to this. For example, the graph of physiological parameters can be uploaded to network by a network interface.

In this embodiment, the graph acquisition device may further include:

a generation unit 703 that is configured to generate the graph of physiological parameters that varies with the progress of reading the multimedia file according to the physiological parameters.

The generation unit 703 is a selectable component, which is shown by dashed frame in FIG. 7. That is, the graph of physiological parameters can be generated in the graph acquisition device 700 or not, for example, the graph acquisition device 700 transmits the obtained physiological parameters into a graph supply device in the following embodiment 4, and the graph of physiological parameters is generated by using the graph supply device, wherein the graph combination device can be e.g. a server.

The first transmission unit 702 can be used for transmitting the graph of physiological parameters when the graph acquisition device includes the generation unit 703.

In this embodiment, the graph of physiological parameters that varies along with the progress of reading the multimedia file can be generated by using any one of the existing methods, but the embodiment of the invention is not limited to this. For example, the graph of physiological parameters obtained by the acquisition unit 701 is normalized and afterwards fitted to form the graph.

It can be seen from the above embodiment that, the graph of physiological parameters that varies along with the progress of reading the multimedia file is obtained to be supplied for a user to select the progress of the multimedia file according to the graph of physiological parameters, so as to read the part in the multimedia file in which the user is interested conveniently and efficiently.

### Embodiment 3

The embodiment of the invention provides an electronic equipment, and FIG. 8 is a structural schematic of the electronic equipment of the embodiment. As shown in FIG. 8, the electronic equipment 800 includes a multimedia reading control device 801 and/or a graph acquisition device 802. Structure and function of the multimedia reading control device 801 are the same with that are recorded in embodiment 1, and structure and function of the graph acquisition device 802 are the same with that are recorded in embodiment 2, and which are not repeated herein.

When the electronic equipment 800 includes the multimedia reading control device 801 and the graph acquisition device 802, the multimedia reading control device 801 reads and controls the multimedia file by using the graph of physiological parameters generated by the graph acquisition device 802.

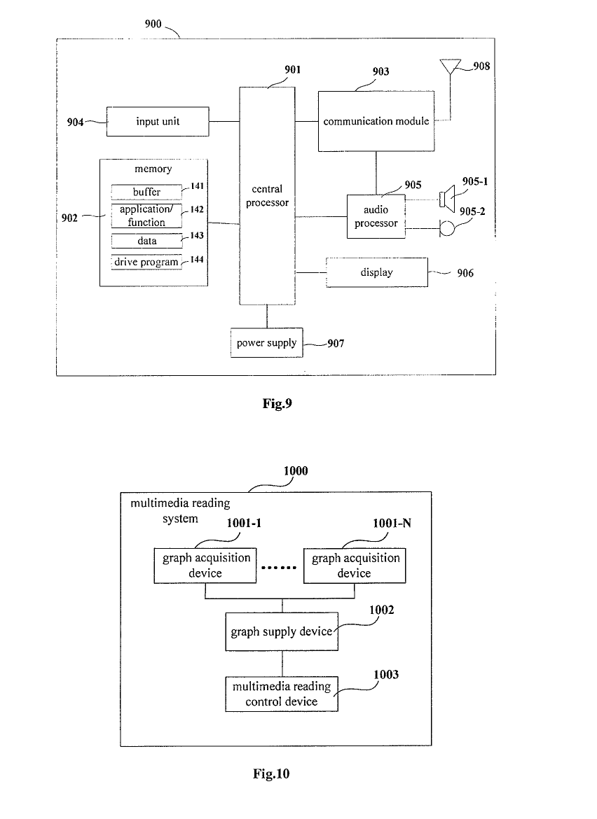

FIG. 9 is a schematic block diagram of system composition of the electronic equipment of the embodiment of the invention. As shown in FIG. 9, the electronic equipment 900 may include a central processor 901 and a memory 902; the memory 902 is coupled into the central processor 902. It is worth noting that this figure is exemplary; it is also possible to use other types of structure to supplement or replace the structure, to realize telecommunication function or other functions.

In this embodiment, functions of the multimedia reading control device can be integrated into the central processor 801. The central processor 901 can be configured for receiving a graph of physiological parameters that varies along with the progress of reading a multimedia file, displaying the graph of physiological parameters, and reading the multimedia file in response to the progress selected according to the graph of physiological parameters; and/or

acquiring physiological parameters of a user when enjoying a multimedia file, so as to be used for generating a graph of physiological parameters that varies with the progress of reading the multimedia file; transmitting the physiological parameters. The reading the multimedia file in response to the progress selected according to the graph of physiological parameters includes:

receiving an instruction for selecting the progress according to the graph of physiological parameters; determining the selected progress according to the instruction; reading the multimedia file according to the selected progress.

In another embodiment, the multimedia reading control device can be configured separately from the central processor 901, for example, the multimedia reading control device can be configured to be a chip connected with the central processor 901, and the function of the multimedia reading control device is achieved by controlling the central processor.

As shown in FIG. 9, the user equipment may further include a communication module 903, an input unit 904, an audio processor 905, a display 906 and a power supply 907. It is worth noting that the user equipment 900 does not have to include all components shown in FIG. 9; in addition, the electronic equipment 900 may further include components that are not shown in FIG. 9 with reference to the prior art.

As shown in FIG. 9, the central processor 901 sometimes is also called a controller or an operation control which may include a microprocessor and other processor devices and/or logic devices. The central processor 901 receives, inputs and controls operation of components of the electronic equipment 900.

The memory 902 can be one or more of e.g. a cache, a flash memory, a hard drive, a removable media, a volatile memory, a nonvolatile memory or other appropriate devices. The above information related to failure can be stored, and besides procedure for implementing relevant information can be stored. In addition, the central processor 901 may implement the procedure stored by the memory 902 to realize information storage or processing or the like. Functions of other components are similar to traditional functions, and thus are not repeated herein. The components of the electronic equipment 900 can be implemented by special hardware, firmware, software or the combination thereof, without departure from the scope of the invention.

It can be seen from the above embodiment that, the graph of physiological parameters varying with the progress of reading the multimedia file is displayed when the multimedia file is read, so that a user selects the progress of the multimedia file according to the graph of physiological parameters, so as to read the part in the multimedia file in which the user is interested conveniently and efficiently.

### Embodiment 4

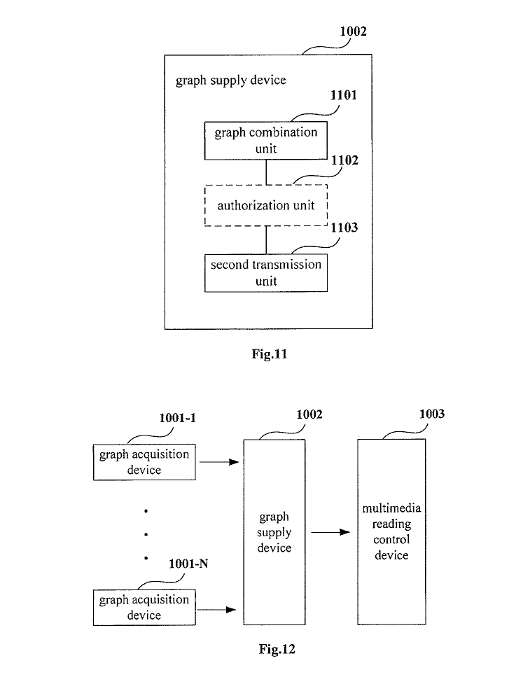

The embodiment of the invention provides a multimedia reading system, and FIG. 10 is a structural schematic of the multimedia reading system 1000. As shown in FIG. 10, the multimedia reading system includes N graph acquisition devices 1001-1, . . . , 1001-N, a graph supply device 1002 and a multimedia reading control device 1003, N is greater than or equal to 2, wherein

structure and function of the graph acquisition devices 1001-1, . . . , 1001-N are the same with that are recorded in embodiment 2, and thus are not repeated herein;

the graph supply device 1002 is used for combining the graphs of physiological parameters generated by the graph generation devices 1001-1, . . . , 1001-N to obtain a combined graph of physiological parameters;

structure and function of the multimedia reading control device 1003 are the same with that are recorded in embodiment 1, and thus are not repeated herein. The multimedia reading control device 1003 receives and displays the graph of physiological parameters that is the combined graph of physiological parameters combined by the graph supply device 1002.

In this embodiment, one of N graph acquisition devices and the multimedia reading control device can be configured in one identical electronic equipment. Structure and function of the electronic equipment are the same with that are recorded in embodiment 3, and thus are not repeated herein.

It can be seen from the above embodiment that, the graph of physiological parameters varying with the progress of reading the multimedia file is displayed when the multimedia file is read, so that a user selects the progress of the multimedia file according to the graph of physiological parameters, so as to read the part in the multimedia file in which the user is interested conveniently and efficiently.

In this embodiment, type of the physiological parameters is the same with that is recorded in embodiment 1, and thus is not repeated herein.

In this embodiment, the graph supply device 1002 is used for combining the graphs of physiological parameters generated by the graph acquisition device 1001-1, . . . , to obtain the combined graph of physiological parameters. The graph supply device 1002 can be e.g. a server, but the embodiment of the invention has no limitation to this. The structure of the graph supply device can be any one of the existing structure, but the embodiment of the invention has no limitation to the structure. Hereinafter the structure of the graph supply device will be described exemplarily.

FIG. 11 is a structural schematic of the graph supply device of the embodiment of the invention. As shown in FIG. 11, the graph supply device 1002 includes a graph combination unit 1101, an authorization unit 1102 and a second transmission unit 1103, wherein

the graph combination unit 1101 is configured to combine at least two graphs of physiological parameters that vary with the progress of reading the multimedia file to obtain a combined graph of physiological parameters;

the authorization unit 1102 is configured to verify a request of transmitting the graph of physiological parameters;

the second transmission unit 1103 is configured to transmit the combined graph of physiological parameters.

In this embodiment, the graph combination unit 1102 may combine at least two graphs of physiological parameters by using any one of the existing methods, but the embodiment of the invention has no limitation to this. For example, the combined graph of physiological parameters can be obtained by the method of calculating a mean value, wherein for one identical progress, a mean value of physiological parameters of the progress of at least two graphs of physiological parameters is calculated, so as to generate the combined graph of physiological parameters according to the mean value of the physiological parameters of the progresses.

In this embodiment, the authorization unit 1102 may be a selectable component and shown by dashed frame in FIG. 11, that is, the combined graph of physiological parameters can be transmitted directly without being verified by the authorization unit. The authorization unit may verify a request of transmitting the graph of physiological parameters by using any one of the existing methods, but the embodiment of the invention has no limitation to this. For example, the authorization unit receives a request of transmitting the graph of physiological parameters from an entertainment company or an individual user and verifies an ID of the entertainment company or the individual user, informs the second transmission unit 1103 to transmit the combined graph of physiological parameters to the entertainment company or the individual user when the ID is in a pre-established database.

In this embodiment, the second transmission unit 1103 may transmit the combined graph of physiological parameters by using any one of the existing methods, but the embodiment of the invention has no limitation to this. For example, transmission is performed in the way of wireless communication.

In this way, accuracy of the graph of physiological parameters can be improved by combining two or more graphs of physiological parameters.

FIG. 12 is a schematic of the multimedia reading system of the embodiment that performs multimedia playing. As shown in FIG. 12, the graph acquisition devices 1001-1, . . . , 1001-N transmits N graphs of physiological parameters to the graph supply device 1002 which combines the received N graphs of physiological parameters and transmits the combined graph of physiological parameters to the multimedia reading control device 1003, which receives the combined graph of physiological parameters and afterwards display the graph of physiological parameters, so that the user can select the progress according to the graph of physiological parameters and read the multimedia file according to the progress selected by the user. The display method of the graph of physiological parameters, and the method of reading the multimedia file according to the progress selected by the user are the same with that are recorded in embodiment 1, and thus are not repeated herein.

In this embodiment, it is also possible for the graph acquisition device to not generate the graph of physiological parameters, but transmit the obtained physiological parameters to the graph combination device directly. The graph combination device generates the graph of physiological parameters according to the physiological parameters and combines the N graphs of physiological parameters.

In this embodiment, types of the multimedia file and the physiological parameters as well as the form of the graph of physiological parameters are the same with that are recorded in embodiment 1, and thus are not repeated herein.

It can be seen from the above embodiment that, the graph of physiological parameters varying with the progress of reading the multimedia file is displayed when the multimedia file is read, so that a user selects the progress of the multimedia file according to the graph of physiological parameters, so as to read the part in the multimedia file in which the user is interested conveniently and efficiently.

### Embodiment 5

The embodiment of the invention provides a multimedia reading control method. The principle of the method to solve problems is similar to the functions of the components of the multimedia reading control device of embodiment 1, therefore specific implementation of the method can be achieved by reference to the implementation of the multimedia reading control device of embodiment 1, with the same contents that are not repeated herein.

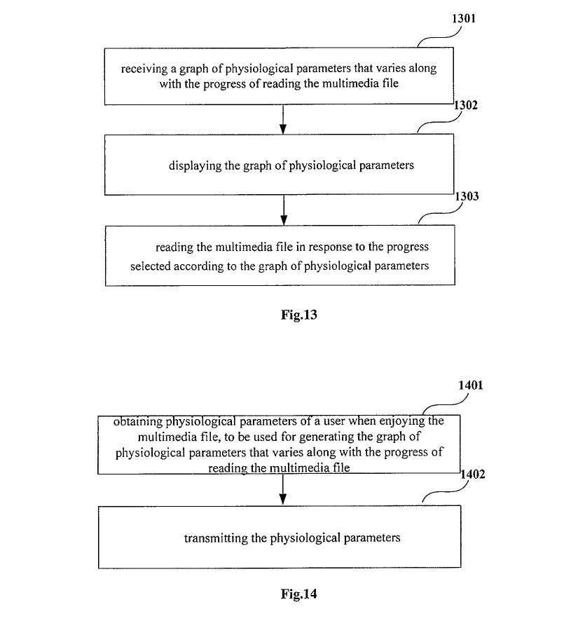

FIG. 13 is a flow chart of the multimedia reading control method of the embodiment of the invention. As shown in FIG. 13, the method includes:

step 1301: receiving a graph of physiological parameters that varies with the progress of reading a multimedia file;

step 1302: displaying the graph of physiological parameters;

step 1303: reading the multimedia file in response to the progress selected according to the graph of physiological parameters.

The types of the multimedia file and the physiological parameters of the embodiment, the method of obtaining the physiological parameters, the method of displaying the graph of physiological parameters and the method of reading the multimedia file according to the progress selected by the user are the same with that are recorded in embodiment 1, and thus are not repeated herein.

It can be seen from the above embodiment that, the graph of physiological parameters varying with the progress of reading the multimedia file is displayed when the multimedia file is read, so that a user selects the progress of the multimedia file according to the graph of physiological parameters, so as to read the part in the multimedia file in which the user is interested conveniently and efficiently.

### Embodiment 6

The embodiment of the invention provides a graph acquisition method. The principle of the method to solve problems is similar to the functions of the components of the graph acquisition device of embodiment 2, therefore specific implementation of the method can be achieved by reference to the implementation of the graph acquisition device of embodiment 2, with the same contents that are not repeated herein.

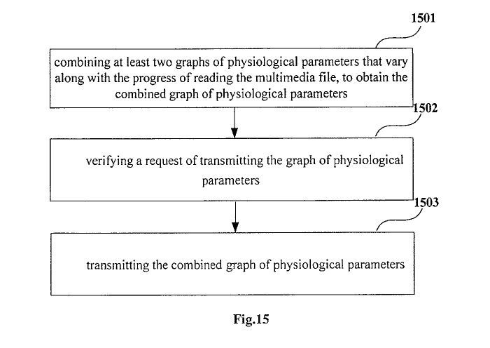

FIG. 14 is a flow chart of the graph generation method of the embodiment of the invention. As shown in FIG. 14, the method includes:

step 1401: acquiring physiological parameters of a user when enjoying a multimedia file, so as to be used for generating a graph of physiological parameters that varies with the progress of reading the multimedia file;

step 1402: transmitting the physiological parameters.

The method of measuring the physiological parameters, the method of generating the graph of physiological parameters and the method of transmitting the graph in the embodiment are the same with that are recorded in embodiment 2, and thus are not repeated herein.

It can be seen from the above embodiment that, the graph of physiological parameters that varies along with the progress of reading the multimedia file is generated to be supplied for a user to select the progress of the multimedia file according to the graph of physiological parameters, so as to read the part in the multimedia file in which the user is interested conveniently and efficiently.

### Embodiment 7

The embodiment of the invention provides a graph supply method. The principle of the method to solve problems is similar to the functions of the components of the graph supply device of embodiment 4, therefore specific implementation of the method can be achieved by reference to the implementation of the graph supply device of embodiment 4, with the same contents that are not repeated herein.

FIG. 15 is a flow chart of the graph supply method of the embodiment of the invention. As shown in FIG. 15, the method includes:

step 1501: combining at least two graphs of physiological parameters that vary with the progress of reading the multimedia file to obtain a combined graph of physiological parameters;

step 1502: verifying a request of transmitting the graph of physiological parameters;

step 1503: transmitting the combined graph of physiological parameters.

In this embodiment, the step 1502 is a selectable step, that is, the combined graph of physiological parameters can be transmitted directly without being verified by the authorization unit.

The method of combining the graphs of physiological parameters, the method of verifying a transmission request and the method of transmitting the graph in the embodiment are the same with that are recorded in embodiment 4, and thus are not repeated herein.

It can be seen from the above embodiment that, the graph of physiological parameters that varies along with the progress of reading the multimedia file is obtained to be supplied for a user to select the progress of the multimedia file according to the graph of physiological parameters, so as to read the part in the multimedia file in which the user is interested conveniently and efficiently.

The above devices and methods of the invention can be achieved by hardware, or can be achieved by the hardware combined with software. The invention relates to such a computer readable program, when the program is executed by a logic component, so that it is possible for the logic component to realize the above devices and components, or to realize the above various methods or steps.

The invention also relates to a storage media for storing the above programs, such as a hard disk, a magnetic disk, an optical disk, a DVD, a flash memory and the like.

The invention is described by combing specific embodiments in the above, but those skilled in the art should understand that, the description is exemplary, and does not limit the protection scope of the invention. Those skilled in the art can make variations and modifications to the invention according to the spirit and principle of the invention, and these variations and modifications shall fall within the scope of the invention.

1. A multimedia reading control device, comprising:
a reception unit configured to receive a graph of physiological parameters that varies with the progress of reading a multimedia file;
a display unit configured to display the graph of physiological parameters;
a response unit configured to read the multimedia file in response to the progress selected according to the graph of physiological parameters.

2. The multimedia reading control device according to claim 1, wherein the response unit comprises:
an instruction receiving unit configured to receive an instruction for selecting the progress according to the graph of physiological parameters;
a determination unit configured to determine the selected progress according to the instruction;
a reading unit configured to read the multimedia file according to the selected progress.

3. The multimedia reading control device according to claim 1, wherein the physiological parameters comprise at least one of heartbeat, pulse, blood pressure, body temperature, a respiration parameter, sound, a focus of eyes, pupil size, facial action, body movements and secretions.

4. The multimedia reading control device according to claim 1, wherein the graph of physiological parameters received by the reception unit is a combined graph of physiological parameters that is obtained by combining at least two graphs of physiological parameters.

5. A graph acquisition device, comprising:
an acquisition unit configured to acquire physiological parameters of a user when enjoying a multimedia file, so as to be used for generating a graph of physiological parameters that varies with the progress of reading the multimedia file;
a transmission unit configured to transmit the physiological parameters.

6. The graph acquisition device according to claim 5, further comprising:
a generation unit configured to generate the graph of physiological parameters that varies with the progress of reading the multimedia file according to the physiological parameters.

7. The graph acquisition device according to claim 5, wherein the physiological parameters comprise at least one of heartbeat, pulse, blood pressure, body temperature, pupil size, facial action, body movements and secretions.

8. An electronic equipment, comprising the multimedia reading control device according to claim 1, and a graph acquisition device comprising
an acquisition unit configured to acquire physiological parameters of a user when enjoying a multimedia file, so as to be used for generating a graph of physiological parameters that varies with the progress of reading the multimedia file; and
a transmission unit configured to transmit the physiological parameters.

9. A graph supply device, comprising:
a graph combination unit configured to combine at least two graphs of physiological parameters that vary with the progress of reading the multimedia file to obtain a combined graph of physiological parameters;
a transmission unit configured to transmit the combined graph of physiological parameters.

10. The graph supply device according to claim 9, further comprising:
an authorization unit configured to verify a request of transmitting the graph of physiological parameters.

11. The graph supply device according to claim 9, wherein the physiological parameters comprise at least one of heartbeat, pulse, blood pressure, body temperature, a respiration parameter, sound, a focus of eyes, pupil size, facial action, body movements and secretions.

12. A multimedia reading control method, comprising:
receiving a graph of physiological parameters that varies with the progress of reading a multimedia file;
displaying the graph of physiological parameters;
reading the multimedia file in response to the progress selected according to the graph of physiological parameters.

13. The multimedia reading control method according to claim 12, wherein reading the multimedia file in response to the progress selected according to the graph of physiological parameters comprises:
receiving an instruction for selecting the progress according to the graph of physiological parameters;
determining the selected progress according to the instruction;
reading the multimedia file according to the selected progress.

14. The multimedia reading control method according to claim 11, wherein the physiological parameters comprise at least one of pulse, blood pressure, body temperature, a respiration parameter, sound, a focus of eyes, pupil size, facial action, body movements and secretions.

15. The multimedia reading control method according to claim 12, wherein the received graph of physiological parameters is a combined graph of physiological parameters that is obtained by combining at least two graphs of physiological parameters.

16. A graph acquisition method, comprising:
acquiring physiological parameters of a user when enjoying a multimedia file, so as to be used for generating a graph of physiological parameters that varies with the progress of reading the multimedia file;
transmitting the graph of physiological parameters.

17. The graph acquisition method according to claim 16, further comprising:
generating the graph of physiological parameters that varies with the progress of reading the multimedia file according to the physiological parameters.

18. The graph acquisition method according to claim 16, wherein the physiological parameters comprise at least one of pulse, blood pressure, body temperature, a respiration parameter, sound, a focus of eyes, pupil size, facial action, body movements and secretions.

19. A graph supply method, comprising:
combining at least two graphs of physiological parameters that vary with the progress of reading the multimedia file to obtain a combined graph of physiological parameters;
transmitting the combined graph of physiological parameters.

20. The graph supply method according to claim 19, further comprising:
verifying a request of transmitting the graph of physiological parameters.

21. (canceled)
